(19) United States
(12) Patent Application Publication
(10) Pub. No.: US 2025/0288389 A1
(43) Pub. Date: Sep. 18, 2025

(54) SURGICAL DEVICE INTEGRATED CONTROL

(71) Applicant: Ayal Willner, Long Beach, CA (US)

(72) Inventor: Ayal Willner, Long Beach, CA (US)

(21) Appl. No.: 18/607,671

(22) Filed: Mar. 18, 2024

(51) Int. Cl. A61B 90/90; A61B 17/00; G16H 40/63

(52) U.S. Cl. CPC A61B 90/90; A61B 17/00; G16H 40/63; A61B 2017/00973

(57) ABSTRACT

An example process may include identifying an object in a field of view, detecting an identifier associated with the object, comparing the identifier to a list of identifiers to identify a corresponding device, and activating a pedal to control the device based on a device profile.

### FIELD

The present invention relates to a system for integrated control of powered surgical instrumentation during a surgical procedure, and more particularly, to an integrated system operated by a surgeon to control various surgical components.

### BACKGROUND

In operating rooms today, many different instruments utilize pedals or other actuator switches. Such devices are called electrosurgical units (ESUs). Often, more than one, even multiple different ESUs are used throughout the course of a surgical procedure. This can lead to a plethora of pedals at the surgeons' feet, making it difficult to operate any of the equipment due to cluttering. It also leads to frustration on the part of the surgeons, nurses, and other operating room personnel, as they struggle to identify the correct pedal under a surgeon's foot at any particular time. The surgeons also struggle to depress the correct pedal switch without activating a different piece of equipment. All of these examples create a hazardous and dangerous environment for the patient.

Accordingly, an improved integrated surgery device control configuration may be beneficial.

### SUMMARY

Certain embodiments of the present invention may provide solutions to the problems and needs in the art that have not yet been fully identified, appreciated, or solved by current surgical device technologies. For example, some embodiments of the present invention pertain to identification of surgical instruments and assignment of the surgical instruments to a particular control mechanism, such as a foot pedal, while the instrument is being used in a surgical procedure.

In an embodiment, a process may include detecting an identifier associated with an object, identifying the object in a field of view, comparing the identifier to a list of identifiers to identify a corresponding device linked to the object, and activating one or more pedals to control the device based on a device profile.

Another embodiment may include a system with one or more pedals, a detection unit configured to detect an identifier associated with an object, identify the object in a field of view, and a processor configured to compare the identifier to a list of identifiers to identify a corresponding device linked to the object, and activate the one or more pedals to control the device based on a device profile.

Another embodiment may include a non-transitory computer readable storage medium configured to store instructions that when executed cause a processor to perform detecting an identifier associated with an object, identifying the object in a field of view, comparing the identifier to a list of identifiers to identify a corresponding device linked to the object, and activating one or more pedals to control the device based on a device profile.

### BRIEF DESCRIPTION OF THE DRAWINGS

In order that the advantages of certain embodiments of the invention will be readily understood, a more particular description of the invention briefly described above will be rendered by reference to specific embodiments that are illustrated in the appended drawings. While it should be understood that these drawings depict only typical embodiments of the invention and are not therefore to be considered to be limiting of its scope, the invention will be described and explained with additional specificity and detail through the use of the accompanying drawings, in which:

FIG. 1 is an architectural diagram illustrating a computing system configured to control operations of operating room equipment according to example embodiments.

FIG. 2 is an example configuration illustrating the visualization array detecting surgical equipment and updating controls according to example embodiments.

FIG. 3 is a flow diagram illustrating an example process of identifying an object in a field of view according to example embodiments.

FIG. 4 is a computer system configured to perform one or more operations corresponding to the example embodiments.

### DETAILED DESCRIPTION

Some embodiments simplify the process of managing various devices in a surgical environment by utilizing a single foot operated actuator (FOA) to operate a plurality of surgical instruments. Surgical instruments may include, but are not limited to, Electrocautery (e.g., Bovie, Suction Bovie), Microdebriders, Radiofrequency ablators/coagulators (Coblator), Bipolar cautery, Ultrasonic aspirators (CUSA), Endoscopic telescope irrigators (Endoscrub), Drill systems (Midas Rex), to name a few. Each surgical instrument, which has its own respective identification (ID)/tag, is detected by an integrated system, thereby allowing the integrated system to load corresponding profile of the ESU foot pedal or foot switch and assign the pedals or footswitches of that ESU to the foot pedals or footswitches of the (FOA). This system may thus turn the FOA into a clone of the OEM foot switch. The FOA may then interact with the actual ESU, via the integrated system to activate the appropriate ESU whose profile has just been selected. In this way, the FOA activates the ESU and its piece of surgical equipment as if the FOA was the OEM foot pedal. In some embodiments, selection of the surgical equipment, and thus the FOA profile, may be performed by touch screen and/or by voice activated selection of the surgical equipment, to name a few.

The integrated system and a corresponding computer application are configured to activate each electrosurgical units (ESUs) using a common FOA. The FOA may include pedals and buttons that are assigned to, and actuate, a particular action associated with the selected ESU. The integrated system may use wireless communication in order for controls to be performed by one actuator at the surgeon's feet without the clutter of many switches and wires. The wireless communication may include bidirectional communication or unidirectional communication. In another embodiment, the integrated system may use wired communication. However, the type of communication is based on system configuration and design.

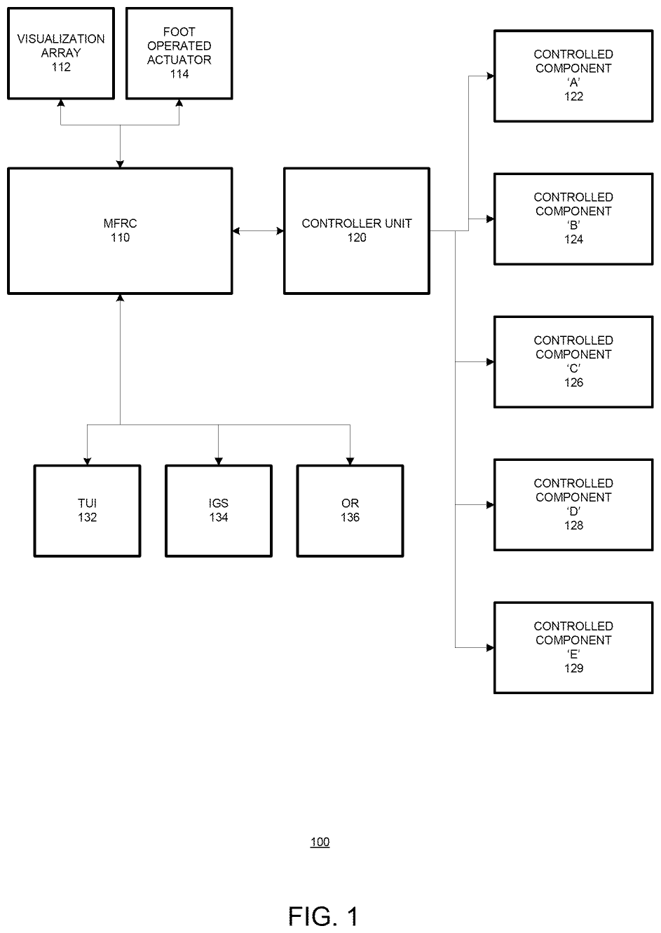

FIG. 1 is an architectural diagram illustrating a computing system 100 configured to control operations of operating room equipment, according to an embodiment of the present invention. In some embodiments, controller unit 120 controls various different components of equipment in the operating room. Equipment, for purposes of explanation, may include, Electrocautery (Bovie, Suction Bovie), Microdebriders, Radiofrequency ablators/coagulators (Coblator), Bipolar cautery, Ultrasonic aspirators (CUSA), Endoscopic telescope irrigators (Endoscrub), and Drill systems (Midas Rex), to name a few. Controller unit 120 may also control other attributes of the environment such as changing the physical environment (e.g., lighting, room temperature, air flow, music, etc.). A software based application operating on a computer may be the main device used as controller unit 120. The configuration may be hardware and software components, including visualization components to identify the “active” handpiece(s) and the corresponding ESU instruments as they are used. Certain ESUs, such as surgical equipment ‘A’ 220, may be identified as they are brought into a field of view (FOV). The ESUs may include those which are often used in surgical environments, i.e., those controlled by peripheral devices such as the foot operated actuator (FOA) 114 in which the pedals and/or any additional actuators or buttons are assigned actions dynamically by controller unit 120. For purposes of explanation, ESUs may include, Electrocautery (Bovie, Suction Bovie), Microdebriders, Radiofrequency ablators/coagulators (Coblator), Bipolar cautery, Ultrasonic aspirators (CUSA), Endoscopic telescope irrigators (Endoscrub), and Drill systems (Midas Rex), to name a few.

Controller unit 120 may interface with various pieces of equipment (e.g., Electrocautery (Bovie, Suction Bovie), Microdebriders, Radiofrequency ablators/coagulators (Coblator), Bipolar cautery, Ultrasonic aspirators (CUSA),

Endoscopic telescope irrigators (Endoscrub), Drill systems (Midas Rex)) and a central computer that may be used to activate certain appropriate instrument equipment on an as needed basis. In these embodiments, certain appropriate equipment may include Electrocautery (Bovie, Suction Bovie), Microdebriders, Radiofrequency ablators/coagulators (Coblator), Bipolar cautery, Ultrasonic aspirators (CUSA), Endoscopic telescope irrigators (Endoscrub), and Drill systems (Midas Rex. A user interface or monitor may be situated near the surgical field, i.e., within reach of the surgeon or assistant that is connected to controller unit 120 and may permit the surgeon and the technician to manage the integrated system. In some embodiments, the user interface or monitor, although not shown, may be a haptic device.

The application may use (bi-directional) wireless communication protocols and/or sensors to identify surgical instruments that are entering a surgical area or the FOV. The ESU handpieces may have wireless communication tags such as an array of reflective dots, radio frequency identifiers (RFID), sensors, QR codes, etc. These wireless tags can be identified by a detection unit (not shown) to identify a particular instrument entering the surgical area or FOV. This may enable the dynamically configured pedal to be used for that ESU handpiece until another ESU handpiece is brought into the FOV of the detection unit.

When a new ESU handpiece is identified as being the next ESU handpiece to be used, the profile of the FOA 114 is changed to operate that next identified ESU equipment The application, in some embodiments, identifies one or more pedals/buttons being activated and then communicates with controller unit 120 to appropriately activate the identified ESU instrument/equipment. Communication with controller unit 120 can be either hardwired or wireless, and as discussed above, may be birectional. The application may display or otherwise communicate with the users (or surgeon) regarding the selected ESU instrument or surgical equipment and the settings on that ESU instrument or surgical equipment (e.g., speed, temperature, gauge, etc.), as well as a graphic indicating the selected function each pedal or button on the FOA 114.

Other room environmental controls may be controlled by the application. This may include, but is not limited to, operating room temperature adjustments, lighting adjustments, and music selections. In one example, when an ESU instrument, such as an electrocoagulation or electro-cautery device, is brought into the surgical area, visualization array 112 identifies when the handpiece associated with the ESU is moving into the field area via sensors, image data, etc., and towards a field center. In this example, controlling unit 120 may identify the handpiece/ESU, and may assign one or more pedals or buttons on FOA 114. In some embodiments, it may be prudent to use two pedals, e.g., one to “Cut” and the other to “Coagulate”. Other FOA buttons may be used to toggle between the “Cut” or “Coagulate” adjustment modes. This may include, but is not limited to, adjustments to power intensity of the selected mode via the various buttons on the FOA (e.g., higher/lower buttons). The application may attempt to inform the operating room personnel verbally, such as by an audio output: “The electrocautery is active at 20 coagulation and zero cutting” or “the coagulation setting has been changed to 12”, when a button selection is made to change a current setting. This way, the surgeon easily identify the active instruments and enabled setting(s) for that particular instrument prior to depressing one of the main pedals on FOA 140.

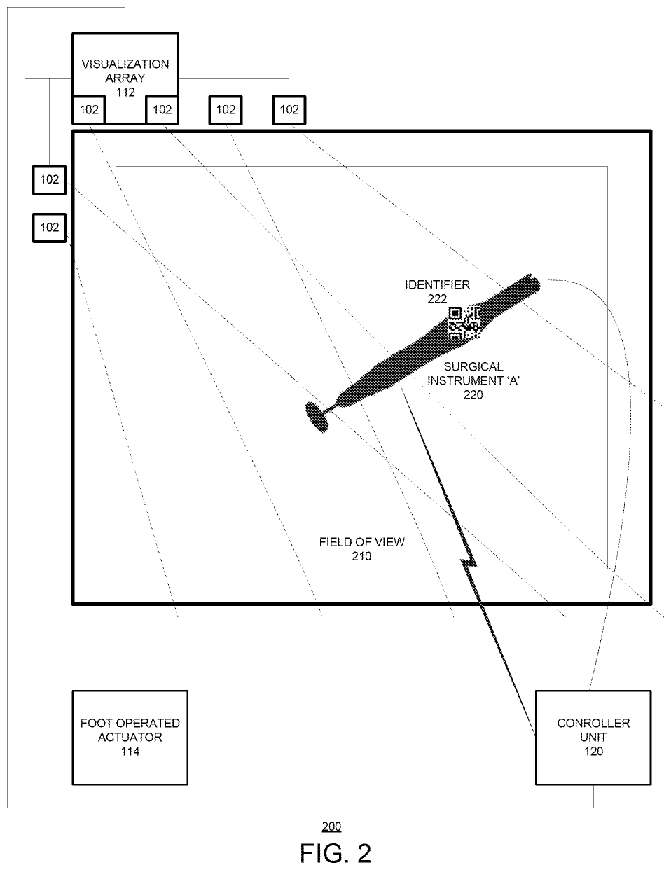

FIG. 2 is a diagram illustrating surgical system 200 configured to detect visualization array/surgical equipment and update controls, according to an embodiment of the present invention. In an embodiment, surgical system 200 includes a touchscreen display (not shown) being positioned near the operating room bed. In some embodiments, the operating room bed may be near FOV 210 and surgical instrument ‘A’ 220 in FIG. 2.

A visualization array 112 (e.g., one or more wired and/or wireless sensors, camera, microphones 102, etc.) are positioned in a manner that identifies objects, movements, sound, etc., which can occur over a defined area of the operating room. In certain embodiments, visualization array 112 may be disposed on a ceiling, overhead lighting configuration and/or a combination of ceiling, wall and floor so that all angles of the operating area are observed by the sensor system. This is generally an area of the operating room bed or station, for example.

In some embodiments, “tags” or identifiers 222, such as a bar code or QR code, is disposed on surgical instruments 220. These surgical instruments 220 may be near the operation area in a FOV 210. The tags may be read by an automated and/or manual reading process, which includes scanning each of the tags one at a time prior to performing surgery. The tags are then uploaded to a database, where the tags are identified as ESU instruments by an assignment database. In a further embodiment, a touch screen user interface adds an ESU instrument to a particular surgical procedure time frame window. Once the ESU instruments are included in a list of surgical instruments, the ESU handpieces are identified by a spoken phrase, an identified bar code, QR code, and/or sensor. This way, the system can “on the fly” reassign the pedals and/or foot switches on the FOA and make the FOA into a “clone” of the OEM foot pedal of the recently identified ESU instrument by visualization array 112.

In some embodiments, surgical system 200 predicts the ESU instrument using a machine learning (ML) and/or artificial intelligence (AI) model. In such embodiments, the ML and/or AI model has memory of which instrument is likely to be used first, second, third, etc. In these embodiments, memory may be based on the surgeon's profile and/or a memory of the procedures associated with the identified procedure (e.g., appendectomy, gall bladder removal, etc.) by controller unit. Components may include a multi-function routing computer (MFRC) 110, a tablet or computer display, a pedal, a controller unit, a handheld scanner or sensor interface where a device may be scanned by its bar code, QR code or other identifying attribute, to name a few. The scanning may be performed automatically by a sensor (not shown). The sensor in some embodiments may identify the ESU instrument moving in and out of the defined area field of view (FOV).

In some embodiments, surgical system 200 includes an array of cameras, sensors, etc., similar to bar code readers or QR code readers. In these embodiments, surgical system 200 identifies, using visualization array 112, the ESU instrument from various different viewing angles. Surgical system 200 may be arranged in a circle or another manner such that when the ESU instrument moves through FOV 210, ESU instrument (or surgical instrument ‘A’ 220) is easily detected from more than one direction. For example, certain wavelengths of light and/or radio signals may be propagated to enable, read and/or identify barcodes, QR codes, a RFID tag, or identify other characteristics associated with a surgical instrument or ESU instrument.

FOA 114 in some embodiments includes one or more pedals. FOA 114 may also have one or more buttons (not shown) associated with the one or more pedals to adjust settings for the one or more pedals. These one or more buttons may adjust the intensity, speed, frequency, etc. The button(s) may be assigned an action by MFRC 110 based on the identified surgical instrument or ESU instrument. Individual pedal/button assignments may be setup in a surgeon profile and recalled when the identified ESU or other controlled component 122-129.

Examples of a controlled components (i.e., surgical devices) may include an electrocautery, suction electrocautery, bipolar cautery, endoscopic microdebrider, endoscopic irrigator, ultra sonic aspirator, electric drill, camera controller, coblator, etc. MFRC 110 may connect via a wireless communication signal to visualization array 112, foot pedal 114, controller unit 120, and an image guidance system (IGS) 134, permitting IGS 134 screen to be used as an interface for MFRC 110. In some embodiments, MFRC 110 may connect to a tablet user interface 132, which is accessible and visible to surgeon at operating room (OR) table 136. Controller unit 120 may be a separate device into which connections are disposed for various ESUs either via wired connections or wirelessly, which would require modules that communicate with the controller and are directly connected to the ESUs. Controller unit 120 may also control a room by operating as a controller for room temperature, lighting, music selection, instruments identifying “tags” and/or stickers with either a bar code or a QR code, which are placed on the instruments, and a handheld reader to permit for each code to be registered to an instrument, to name a few.

In some embodiments, MFRC 110 receives data input from visualization array 112, and determines which ESU instrument handpiece is being used, and reconfigures FOA 114 pedals and buttons based on the pedals and button functions of the ESUs OEM foot pedal. Then, when the pedal on FOA 114 is actuated, MFRC 110 detects the actuation, and directs the controlling unit 120 to activate the appropriate function on the appropriate ESU. MFRC 110 may be located in a “safer” part of the OR, such as a cabinet, while the control box would be near the ESUs themselves. If the connection between the control box and the ESUs is wireless, then the controller box ('controller unit') 120 may be integrated into MFRC 110 and both can be located in a “safer” area of the OR.

During operation, an instrument (ESU) and/or controlled component 122-129 entering the FOV 210 may be identified by the visualization array 112 via one or more sensors 102. Sensors 102 may include cameras, universal product code (UPC) readers, radio frequency (RF) antennae, etc. In certain embodiments, instrument (ESU) or controlled component 122-129 may also be identified by one or more identifiable indicia causing the selected device to enable a change on the screen of the monitor to indicate one or more of, the type of instrument being used, a default action of each pedal or button on the FOA 114, one or more adjustment options, a capability to store a surgeon's personal preferences, an ability to “learn” a surgeon's personal preferences, ability to interact via touch screen and/or voice activation, etc.

There may be restrictions on what role a person in the OR can activate on the control system. For example, a surgeon, nurse, etc., may each have certain restrictions to certain aspects based on predetermined decisions. Some devices may be controlled by FOA 114 while others are voice activated, which is useful when two instruments are in the FOV and both are currently active. The array of scanners or sensors 112 may be an add-on unit, which attaches to the surgical system or may be a free standing array. In one example, when a device is detected, a list, along with visual icons, may be available, and when the reader reads the code, the tagged instrument is assigned to a particular ESU. This way, the tags are attached to the actual instrument that is known to the system and registered to that instrument for recall purposes when the identifier is detected.

When an instrument is detected in the FOV and is moving toward an exact surgical area within the FOV, FOA 114 pedals are assigned to controls for that particular instrument. The surgeon, in this example, is notified by a notification made by the system that the newly detected ESU instrument is the active instrument, along with its settings. MFRC 110 may receive the scan information from visualization array 112 and identify a corresponding tag and instrument since each tag has been registered to a particular ESU instrument. When the ESU instrument (or surgical instrument 220) is no longer in the FOV, MFRC may reassign FOA 114 pedals to activate a new ESU instrument that has recently entered the FOV. MFRC 110 may communicate with TUI 132 to present appropriate graphics and functional information regarding which device is being used by the surgeon at a particular time. MFRC 110 may inform the surgeon via automated speech as to the currently active ESU instrument and/or when that device is activated for use by FOA 114.

For any peripheral changes, such as room temperature, music, etc., TUI 132 may communicate with MFRC 110 to submit the request for changes, MFRC 110 communicates with the appropriate devices, and then the modifications are displayed on tablet user interface (TUI) 132.

In one example, surgical instrument 220 that is entering into the FOV 210, and to which the pedal 114 will be assigned, is announced, along with its settings as an audio message. This notification can be heard by the surgeon to confirm the appropriate instrument choice is selected at a particular time and prior to the surgeon attempting to control the instrument ESU.

Fewer pedals decreases the likelihood of inadvertent activation of a particular instrument. Visualization of the active instrument on TUI 132, along with visualization of the foot operated actuator and the function of the various pedals and buttons on FOA 114, provides users with assurance that a particular device is recognized and being used at a particular time. The assurance reduces ambiguity and increases confidence in the operating room under high pressure circumstances.

As discussed above, controller unit 120 receives instructions from MFRC 110 and activates actions of a particular ESU to which it is attached. In another embodiment, a “mini” controller box is attached to each controlled ESU and MFRC 110 may communicate with each “mini” controller box directly. In either case, controller unit 120 is not intended to communicate directly with FOA 114, as that may make controller unit 120 too big and with too much hardware to withstand being thrown around the OR. In these embodiments, all communication may pass through MFRC 110, either through controller box 120, which is hardwired to all the ESUs, or directly through each “mini” controller box via direct wireless communication with MFRC 110. This way, MFRC 110 is kept in a safe place, and thus, does not need to be “hardened”.

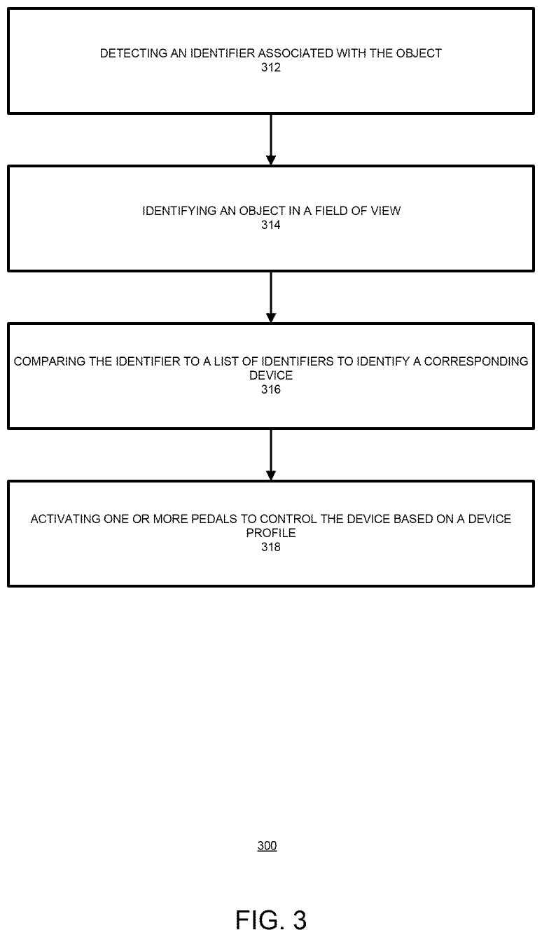

FIG. 3 is a flow diagram illustrating a process 300 of identifying an object in a FOV, according to an embodiment of the present invention. In some embodiments, process 300 may begin at 312 with detecting an identifier associated with the object. Upon detecting the identifier, an object in a FOV is identified at 314. At 316, process 300 includes comparing the identifier to a list of identifiers to identify a corresponding device. At 318, process 300 includes activating one or more pedals to control the device based on a device profile.

The example process may also include receiving image data corresponding to one or more of the identifier and the object via a visualization array disposed above the field of view, determining the object is a surgical instrument, determining the identifier is one or more of a bar code, a quick response (QR) code and a serial number, and forwarding the identifier to a data store comprising the list of identifiers, and identifying a match between the identifier and one or more identifiers in the list of identifiers. Once the code is identified it may be sent to a data store to query other identifiers or codes. The process may also include retrieving content comprising one or more of a device name and a device image, and displaying the content on a display. The device profile may include one or more assignments to assign to the one or more pedals, wherein the one or assignments comprise a speed, a type of movement, and a temperature control. Each pedal and/or button (i.e., 1, 2. . . . N, pedals/buttons) may be an actuator that can be programmed by information from the device profile, such as increase, decrease, forward, reverse, fast, slow, power, no temperature change, etc. The process may also include outputting audio which identifies information regarding which of the one or more pedals is assigned to a respective one or more control functions associated with the device.

Another example embodiment may include a system such as number of units and computers and/or sub-devices including one or more pedals and a detection unit configured to detect an identifier associated with an object, identify the object in a field of view, and a processor configured to compare the identifier to a list of identifiers to identify a corresponding device linked to the object, and activate the one or more pedals to control the device based on a device profile.

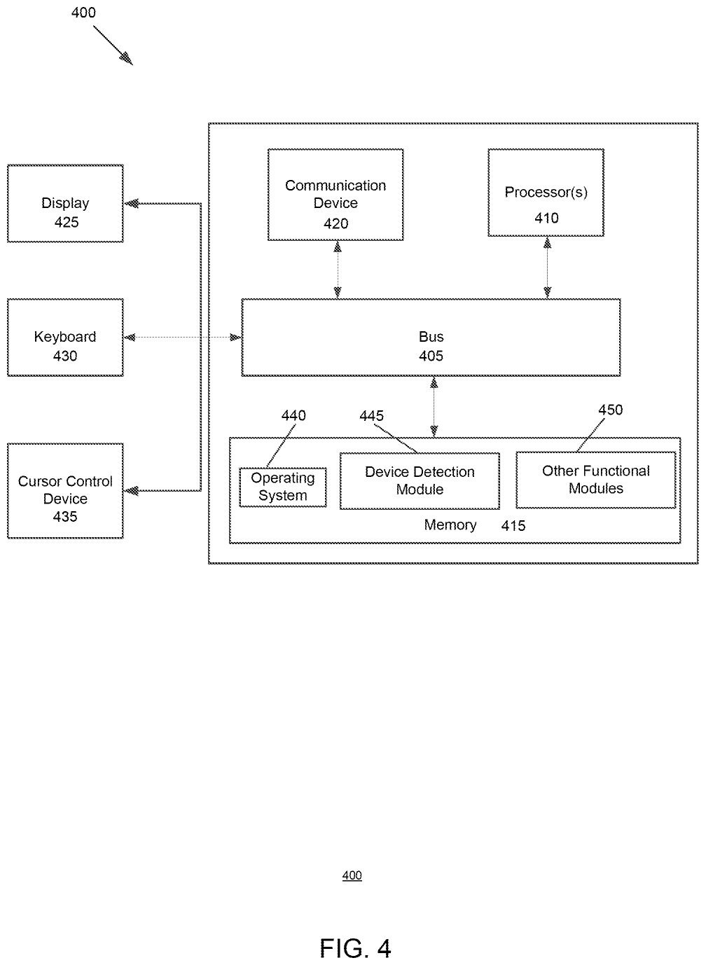

FIG. 4 is a block diagram illustrating a computer system 400 configured to perform one or operations, according to an embodiment of the present invention. In FIG. 4, the architectural diagram illustrates a computing system 400 configured to detect devices in a FOV and activate a controller, such as a foot pedal to control the device once the device is identified, according to an embodiment of the present invention. In some embodiments, computing system 400 may be one or more of the computing systems depicted and/or described herein. Computing system 400 includes a bus 405 or other communication mechanism for communicating information, and processor(s) 410 coupled to bus 405 for processing information. Processor(s) 410 may be any type of general or specific purpose processor, including a Central Processing Unit (CPU), an Application Specific Integrated Circuit (ASIC), a Field Programmable Gate Array (FPGA), a Graphics Processing Unit (GPU), multiple instances thereof, and/or any combination thereof. Processor(s) 410 may also have multiple processing cores, and at least some of the cores may be configured to perform specific functions. Multi-parallel processing may be used in some embodiments. In certain embodiments, at least one of processor(s) 410 may be a neuromorphic circuit that includes processing elements that mimic biological neurons. In some embodiments, neuromorphic circuits may not require the typical components of a Von Neumann computing architecture.

Computing system 400 further includes a memory 415 for storing information and instructions to be executed by processor(s) 410. Memory 415 can be comprised of any combination of Random Access Memory (RAM), Read Only Memory (ROM), flash memory, cache, static storage such as a magnetic or optical disk, or any other types of non-transitory computer-readable media or combinations thereof. Non-transitory computer-readable media may be any available media that can be accessed by processor(s) 410 and may include volatile media, non-volatile media, or both. The media may also be removable, non-removable, or both.

Additionally, computing system 400 includes a communication device 420, such as a transceiver, to provide access to a communications network via a wireless and/or wired connection. In some embodiments, communication device 420 may be configured to use Frequency Division Multiple Access (FDMA), Single Carrier FDMA (SC-FDMA), Time Division Multiple Access (TDMA), Code Division Multiple Access (CDMA), Orthogonal Frequency Division Multiplexing (OFDM), Orthogonal Frequency Division Multiple Access (OFDMA), Global System for Mobile (GSM) communications, General Packet Radio Service (GPRS), Universal Mobile Telecommunications System (UMTS), cdma2000, Wideband CDMA (W-CDMA), High-Speed Downlink Packet Access (HSDPA), High-Speed Uplink Packet Access (HSUPA), High-Speed Packet Access (HSPA), Long Term Evolution (LTE), LTE Advanced (LTE-A), 802.11x, Wi-Fi, Zigbee, Ultra-WideBand (UWB), 802.16x, 802.15, Home Node-B (HnB), Bluetooth, Radio Frequency Identification (RFID), Infrared Data Association (IrDA), Near-Field Communications (NFC), fifth generation (5G), New Radio (NR), any combination thereof, and/or any other currently existing or future-implemented communications standard and/or protocol without deviating from the scope of the invention. In some embodiments, communication device 420 may include one or more antennas that are singular, arrayed, phased, switched, beamforming, beamsteering, a combination thereof, and or any other antenna configuration without deviating from the scope of the invention.

Processor(s) 410 are further coupled via bus 405 to a display 425, such as a plasma display, a Liquid Crystal Display (LCD), a Light Emitting Diode (LED) display, a Field Emission Display (FED), an Organic Light Emitting Diode (OLED) display, a flexible OLED display, a flexible substrate display, a projection display, a 4K display, a high definition display, a Retina® display, an In-Plane Switching (IPS) display, or any other suitable display for displaying information to a user. Display 425 may be configured as a touch (haptic) display, a three dimensional (3D) touch display, a multi-input touch display, a multi-touch display, etc. using resistive, capacitive, surface-acoustic wave (SAW) capacitive, infrared, optical imaging, dispersive signal technology, acoustic pulse recognition, frustrated total internal reflection, etc. Any suitable display device and haptic I/O may be used without deviating from the scope of the invention.

A keyboard 430 and a cursor control device 435, such as a computer mouse, a touchpad, etc., are further coupled to bus 405 to enable a user to interface with computing system. However, in certain embodiments, a physical keyboard and mouse may not be present, and the user may interact with the device solely through display 425 and/or a touchpad (not shown). Any type and combination of input devices may be used as a matter of design choice. In certain embodiments, no physical input device and/or display is present. For instance, the user may interact with computing system 400 remotely via another computing system in communication therewith, or computing system 400 may operate autonomously.

Memory 415 stores software modules that provide functionality when executed by processor(s) 410. The modules include an operating system 450 for computing system 400. The modules further include a device detection module 445 that is configured to perform all or part of the processes described herein or derivatives thereof. Computing system 400 may include one or more additional functional modules 450 that include additional functionality.

One skilled in the art will appreciate that a “system” could be embodied as a server, an embedded computing system, a personal computer, a console, a personal digital assistant (PDA), a cell phone, a tablet computing device, a quantum computing system, or any other suitable computing device, or combination of devices without deviating from the scope of the invention. Presenting the above-described functions as being performed by a “system” is not intended to limit the scope of the present invention in any way, but is intended to provide one example of the many embodiments of the present invention. Indeed, methods, systems, and apparatuses disclosed herein may be implemented in localized and distributed forms consistent with computing technology, including cloud computing systems.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, include one or more physical or logical blocks of computer instructions that may, for instance, be organized as an object, procedure, or function. Nevertheless, the executables of an identified module need not be physically located together, but may include disparate instructions stored in different locations that, when joined logically together, comprise the module and achieve the stated purpose for the module. Further, modules may be stored on a computer-readable medium, which may be, for instance, a hard disk drive, flash device, RAM, tape, and/or any other such non-transitory computer-readable medium used to store data without deviating from the scope of the invention.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

The process steps performed in FIG. 3 may be performed by a computer program, encoding instructions for the processor(s) to perform at least part of the process(es) described in FIG. 3, in accordance with embodiments of the present invention. The computer program may be embodied on a non-transitory computer-readable medium. The computer-readable medium may be, but is not limited to, a hard disk drive, a flash device, RAM, a tape, and/or any other such medium or combination of media used to store data. The computer program may include encoded instructions for controlling processor(s) of a computing system (e.g., processor(s) 410 of computing system 400 of FIG. 4) to implement all or part of the process steps described in FIG. 3, which may also be stored on the computer-readable medium.

The computer program can be implemented in hardware, software, or a hybrid implementation. The computer program can be composed of modules that are in operative communication with one another, and which are designed to pass information or instructions to display. The computer program can be configured to operate on a general purpose computer, an ASIC, or any other suitable device.

It will be readily understood that the components of various embodiments of the present invention, as generally described and illustrated in the figures herein, may be arranged and designed in a wide variety of different configurations. Thus, the detailed description of the embodiments of the present invention, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, reference throughout this specification to “certain embodiments,” “some embodiments,” or similar language means that a particular feature, structure, or characteristic described in connection with the embodiment is included in at least one embodiment of the present invention. Thus, appearances of the phrases “in certain embodiments,” “in some embodiment,” “in other embodiments,” or similar language throughout this specification do not necessarily all refer to the same group of embodiments and the described features, structures, or characteristics may be combined in any suitable manner in one or more embodiments.

It should be noted that reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

1. A method comprising:
detecting an identifier associated with an object;
identifying the object in a field of view;
comparing the identifier to a list of identifiers to identify a corresponding device linked to the object; and
activating one or more pedals to control the device based on a device profile.

2. The method of claim 1, comprising
receiving image data corresponding to one or more of the identifier and the object via a visualization array disposed above the field of view.

3. The method of claim 1, comprising
determining the object is a surgical instrument.

4. The method of claim 1, comprising
determining the identifier is one or more of a bar code, a quick response (QR) code and a serial number; and
forwarding the identifier to a data store comprising the list of identifiers; and
identifying a match between the identifier and one or more identifiers in the list of identifiers.

5. The method of claim 4, comprising
retrieving content comprising one or more of a device name and a device image; and
displaying the content on a display.

6. The method of claim 1, wherein the device profile comprises one or more assignments to assign to the one or more pedals, wherein the one or assignments comprise a speed, a type of movement, and a temperature control.

7. The method of claim 1, comprising
outputting audio which identifies information regarding which of the one or more pedals is assigned to a respective one or more control functions associated with the device.

8. A system comprising:
one or more pedals;
a detection unit configured to
detect an identifier associated with an object;
identify the object in a field of view; and
a processor configured to
compare the identifier to a list of identifiers to identify a corresponding device linked to the object; and
activate the one or more pedals to control the device based on a device profile.

9. The system of claim 8, comprising
a visualization array; and
a receiver configured to receive image data corresponding to one or more of the identifier and the object via the visualization array disposed above the field of view.

10. The system of claim 8, wherein the processor is further configured to determine the object is a surgical instrument.

11. The system of claim 8, wherein the processor is further configured to
determine the identifier is one or more of a bar code, a quick response (QR) code and a serial number; and
forward the identifier to a data store comprising the list of identifiers; and
identify a match between the identifier and one or more identifiers in the list of identifiers.

12. The system of claim 11, wherein the processor is further configured to
retrieve content comprising one or more of a device name and a device image; and
display the content on a display.

13. The system of claim 8, wherein the device profile comprises one or more assignments to assign to the one or more pedals, wherein the one or assignments comprise a speed, a type of movement, and a temperature control.

14. The system of claim 8, wherein the processor is further configured to
output audio which identifies information regarding which of the one or more pedals is assigned to a respective one or more control functions associated with the device.

15. A non-transitory computer readable storage medium configured to store instructions that when executed cause a processor to perform:
detecting an identifier associated with an object;
identifying the object in a field of view;
comparing the identifier to a list of identifiers to identify a corresponding device linked to the object; and
activating one or more pedals to control the device based on a device profile.

16. The non-transitory computer readable storage medium of claim 15, wherein the processor is further configured to perform:
receiving image data corresponding to one or more of the identifier and the object via a visualization array disposed above the field of view.

17. The non-transitory computer readable storage medium of claim 15, wherein the processor is further configured to perform:
determining the object is a surgical instrument.

18. The non-transitory computer readable storage medium of claim 15, wherein the processor is further configured to perform:
determining the identifier is one or more of a bar code, a quick response (QR) code and a serial number; and
forwarding the identifier to a data store comprising the list of identifiers; and
identifying a match between the identifier and one or more identifiers in the list of identifiers.

19. The non-transitory computer readable storage medium of claim 18, wherein the processor is further configured to perform:
retrieving content comprising one or more of a device name and a device image; and
displaying the content on a display.

20. The non-transitory computer readable storage medium of claim 15, wherein the device profile comprises one or more assignments to assign to the one or more pedals, wherein the one or assignments comprise a speed, a type of movement, and a temperature control.
